(19) United States
(12) Patent Application Publication
(10) Pub. No.: US 2025/0041430 A1
(43) Pub. Date: Feb. 6, 2025

(54) CONJUGATE COMPOUND AND METHOD FOR PRODUCING CONJUGATE COMPOUND

(71) Applicant: NIPPON SHOKUBAI CO., LTD., Osaka-shi, Osaka (JP)

(72) Inventors: Masato IMASE, Suita-shi, Osaka (JP); Yuki KISHIMOTO, Suita-shi, Osaka (JP); Takeshi BABA, Suita-shi, Osaka (JP)

(21) Appl. No.: 18/716,339

(22) PCT Filed: Dec. 13, 2022
(86) PCT No.: PCT/JP2022/045820

§ 371 Date: Jun. 4, 2024

(30) Foreign Application Priority Data
Dec. 15, 2021 (JP) 2021-202991

(51) Int. Cl. A61K 47/58

(52) U.S. Cl. CPC A61K 47/58

(57) ABSTRACT

A conjugate compound is capable of inhibiting complement activation. The conjugate compound contains: a polymer (A) which has a constitutional unit derived from a monomer (a) having two or more hydroxyl groups in the molecule and having 2-10 carbon atoms constituting the side chain among the carbon atoms of the constitutional unit; and a component (B) containing at least one selected from the group consisting of an amino acid, a polypeptide, a protein, a nucleoside, a nucleotide, and a nucleic acid.

### TECHNICAL FIELD

The present invention relates to a conjugate compound and a method for producing a conjugate compound. The conjugate compound of the present invention can be suitably used as a pharmaceutical additive or a pharmaceutical.

### BACKGROUND ART

In recent years, a preparation (DDS preparation) based on a drug delivery system (DDS) has been actively developed. In recent DDS preparations, polyethylene glycol (PEG) has been widely used for the purpose of improving the blood (internal) residence time and the stability of the preparation.

For example, PEG-modified liposomes obtained by modifying liposomes or high molecular weight micelles with PEG are used as long-term blood-retaining liposomes as drug carriers, a preparation containing doxorubicin (Doxil (registered trademark)), and the like have been clinically used.

PEG is highly flexible due to the simple skeletal structure thereof. Since PEG has the property of being able to hydrate many water molecules, a thick hydrated layer is formed on the surface layer of the particles by modifying the drug particles and carriers with PEG.

It has been known that this hydrated layer suppresses the interaction with serum proteins and cells, and as a result, the blood (internal) residence time of the drug is greatly extended (stealthization).

For example, Patent Literature 1 describes a method of producing a PEGylated lactoferrin complex, wherein a linear polyethylene glycol (PEG) or a modified product thereof is covalently bonded to lactoferrin via an amide bond, the method including a step of reacting a reaction liquid containing lactoferrin and a linear PEG derivative having a para-nitrophenyl group under conditions that allow formation of an amide group between the para-nitrophenyl group and the lactoferrin, and describes that the lactoferrin's important bioactivity based on the iron binding ability is maintained, and the complex has resistance to proteases such as pepsin by the binding to the linear PEG derivative, so that the complex has a long in vivo lifetime and can exhibit the bioactivity for a long time in the body.

Patent Literature 2 describes that polyethylene glycolated human interferon α2b having the following structure, which is obtained by linking polyethylene glycol (YPEG) having a Y-type branched structure with human interferon α2b (IFN-α2b), is used for the production of a pharmaceutical composition used for the treatment of viral infections such as hepatitis C.

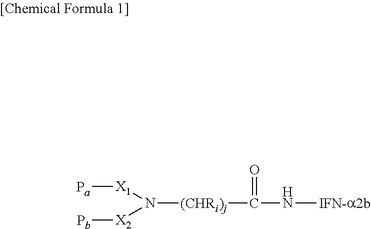

wherein Pa and Pb are the same or different polyethylene glycols, j is an integer of 1 to 12, Ri is hydrogen, a substituted or unsubstituted C1-12 alkyl group, a substituted aralkyl group, an aryl group, or a heteroalkyl group, X₁ and X₂ are each independently a linking group, X₁ is (CH₂)ₙ, X₂ is a group selected from the group consisting of (CH₂)ₙ, (CH₂)ₙOCO, (CH₂)ₙNHCO, and (CH₂)ₙCO, n is an integer of 1 to 10, and YPEG is an amide bond with an ε-amino group on a side chain of lysine of hIFN-α2b corresponding to the position 134 of SEQ ID NO: 1 and is linked to IFN-α2b.

### CITATION LIST

### Patent Literatures

Patent Literature 1: WO 2009/113743 A
Patent Literature 2: JP 2010-538022 A

### SUMMARY OF INVENTION

However, in recent years, in a PEGylated pharmaceutical modified with PEG, it has been reported that an adverse immune reaction associated with unintended complement activation in the body is caused, and a pharmacological effect associated with frequent administration is reduced. Therefore, a compound that can suppress complement activation and replaces the PEGylated pharmaceutical has been strongly desired.

The present inventors have conducted studies in view of problems as described above and have found, as s compound substituted for the PEGylated pharmaceutical, a conjugate compound of a polymer (A) having a constitutional unit derived from a monomer (a) having two or more hydroxyl groups and having 2 to 10 carbon atoms constituting a side chain among carbon atoms of the constitutional unit, and a component (B) containing at least one selected from the group consisting of an amino acid, a polypeptide, a protein, a nucleoside, a nucleotide, and a nucleic acid, thereby completing the present invention.

### BRIEF DESCRIPTION OF DRAWINGS

FIG. 1 a view when Conjugate 1 described in Example 1 is subjected to polyacrylamide electrophoresis (SDS-PAGE) and then subjected to staining or the like, and gel photographing is performed.

FIG. 2 results of evaluating complement activation of Conjugates 1 to 4 described in Example 1, a conjugate using maleimide PEG instead of Polymer 1 of Example 1, and BSA.

FIG. 3 results of analyzing Conjugate 2 described in Example 1 by size exclusion chromatography.

FIG. 4 results of a nucleic acid degradation test of Conjugate 5 described in Example 2 and an unmodified oligonucleic acid.

FIG. 5 results of evaluating complement activation of Conjugates 5 and 6 described in Example 2 and a conjugate using maleimide PEG instead of Polymer 1 of Example 1.

FIG. 6 results of a cytotoxicity test using Polymer 1, Polymer 2, Conjugates 1 and 4 described in Example 1, Conjugate 5 described in Example 2, and Conjugate 7 described in Example 3.

### DESCRIPTION OF EMBODIMENTS

A conjugate compound of the present disclosure is characterized by being formed of a polymer (A) having a constitutional unit derived from a monomer (a) having two or more hydroxyl groups and having 2 to 10 carbon atoms constituting a side chain among carbon atoms of the constitutional unit, and a component (B) containing at least one selected from the group consisting of an amino acid, a polypeptide, a protein, a nucleoside, a nucleotide, and a nucleic acid. According to the conjugate compound of the present disclosure, there is provided a novel conjugate compound modified with a compound other than PEG.

The conjugate compound of the present disclosure is obtained by synthesizing a polymer (A) having a functional group introduced into a terminal, and allowing the polymer (A) to act on a component (B) containing at least one selected from the group consisting of an amino acid, a polypeptide, a protein, a nucleoside, a nucleotide, and a nucleic acid. The functional group of the polymer (A) having a functional group introduced at a terminal may be introduced by a polymerization reaction, or may be introduced by further reacting with a compound having a functional group after polymerization. One feature of the conjugate compound of the present disclosure is that the conjugate compound is bonded by a chemo-selective reaction between the polymer (A) and the component (B) containing at least one selected from the group consisting of an amino acid, a polypeptide, a protein, a nucleoside, a nucleotide, and a nucleic acid.

Hereinafter, preferred embodiments of the present invention will be described. The present invention is not limited only to the following embodiments. A combination of two or more of individual preferred embodiments of the present invention described below is also a preferred embodiment of the present invention.

In the present specification, the phrase “X to Y” indicating a range means “X or more and Y or less”, and “weight” is synonymous with “mass”. In the present specification, “(meth)acrylate” means acrylate or methacrylate, “(meth)acryl” means acryl or methacryl, and acrylate and methacrylate may be used singly respectively, or may be used in combination. Unless otherwise specified, operations and measurements of physical properties and the like are measured under the conditions of room temperature (20 to 25° C.)/relative humidity of 40 to 50%.

### <Polymer (A)>

The constitutional unit derived from a monomer (a) having two or more hydroxyl groups and having 2 to 10 carbon atoms constituting a side chain among carbon atoms of the constitutional unit of the present disclosure means a unit in which one of polymerizable unsaturated double bonds of the monomer (a) having two or more hydroxyl groups and having 2 to 10 carbon atoms constituting a side chain among carbon atoms of the constitutional unit is opened by polymerization to form a part of the polymer. The constitutional unit derived from the monomer (a) may be a constitutional unit formed by another production method as long as it has the same structure as the constitutional unit formed by polymerization of the monomer (a) as described above. In the present specification, unless otherwise specified, the constitutional unit derived from the monomer (a) refers to a constitutional unit derived from one molecule of the monomer (a), and does not refer to the entire constitutional unit contained in the polymer. A constitutional unit derived from a monomer (b) described below is also interpreted in the same manner as the monomer (a) described above.

The monomer (a) having two or more hydroxyl groups and having 2 to 10 carbon atoms constituting a side chain among carbon atoms of the constitutional unit of the present disclosure is preferably a vinyl monomer and more preferably a (meth)acrylic monomer. The monomer (a) may be a monofunctional monomer or a polyfunctional monomer, but preferably includes a monofunctional monomer and more preferably is composed of a monofunctional monomer.

The number of hydroxyl groups contained in the molecule of the monomer (a) is 2 or more, and for example, the number of hydroxyl groups is 2 to 8, 2 to 6, and 2 to 4.

As the monomer (a) of the present disclosure, (meth)acrylates such as glycerin mono(meth)acrylate (also known as: 2,3-dihydroxypropyl (meth)acrylate), 1,2-dihydroxyethyl (meth)acrylate, 2,2-dihydroxyethyl (meth)acrylate, dihydroxybutyl (meth)acrylate, trimethylolpropane mono(meth)acrylate, pentaerythritol mono(meth)acrylate, and dipentaerythritol mono(meth)acrylate are suitably used. Among them, the monomer (a) preferably includes glycerol monoacrylate (GLMA) and/or glycerol monomethacrylate (GLMMA), from the viewpoint of ease of industrial availability, high reactivity, high bondability between the obtained polymer and the component (B), an effect of suppressing complement activation, and the like. For example, the content of glycerol monoacrylate (GLMA) and/or glycerol monomethacrylate (GLMMA) in the monomer (a) is preferably 20 parts by mass or more (upper limit: 100 parts by mass), 40 parts by mass or more, 60 parts by mass or more, 80 parts by mass or more, 90 parts by mass or more, or 95 parts by mass or more in this order. By polymerizing these monomers (a), the ethylenic double bond contained in the monomer (a) is cleaved to generate a constitutional unit. Only one kind of the monomer (a) may be used singly, or two or more kinds thereof may be used in combination.

The number of carbon atoms constituting a side chain among carbon atoms of the constitutional unit derived from the monomer (a) is 2 to 10, but in the present specification, the term “side chain” refers to a portion other than the main chain. The phrase “2 to 10 carbon atoms constituting a side chain” refers to (the sum of) the number of carbon atoms in the entire side chain (the entire (four) groups bonded to the carbon atoms of the main chain). The “main chain” means a chain of continuously bonded carbon atoms in a polymer consisting of a series of constitutional units, which has the largest number of carbon atoms. As described above, the number of carbon atoms constituting a side chain among carbon atoms of the constitutional unit derived from the monomer (a) is 2 to 10, and the number of carbon atoms is preferably 3 to 8 and more preferably 4 to 6. The side chain of the constitutional unit derived from the monomer (a) may be an unsubstituted or substituted alkyl group having 2 to 10 carbon atoms, the substituent may be a hydroxyl group, and two or more hydroxyl groups as the substituent may be contained.

The constitutional unit derived from the monomer (a) of the present disclosure preferably includes a constitutional unit represented by the following General Formula (5).

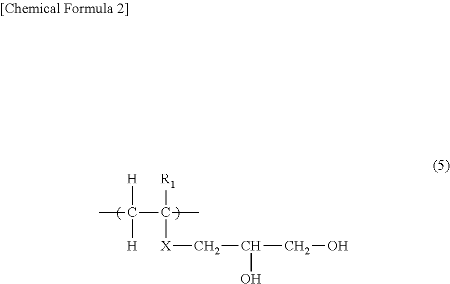

In General Formula (5), R₁ represents a hydrogen atom or a methyl group, and X represents —C(═O)—O—, —C(═O)—NH—, —O—, —CH₂O—, or —CH₂CH₂O—, preferably —C(═O)—O—. The content of the constitutional unit represented by General Formula (5) in the constitutional unit of the monomer (a) is, for example, preferably 20 parts by mass or more (upper limit: 100 parts by mass), 40 parts by mass or more, 60 parts by mass or more, 80 parts by mass or more, 90 parts by mass or more, or 95 parts by mass or more in this order.

Among the constitutional units represented by the above General Formula (5), those in which R₁ is a hydrogen atom and X is —C(═O)—O— are derived from glycerol monoacrylate (GLMA) as the monomer (a). Among the constitutional units represented by the above Chemical Formula (1), those in which R₁ is a methyl group and X is —C(═O)—O— are derived from glycerol monomethacrylate (GLMMA) as the monomer (a).

The content of the constitutional unit derived from the monomer (a) per 100 parts by mass of the polymer (A) of the present disclosure is preferably 5 parts by mass or more (upper limit: 100 parts by weight), more preferably 20 parts by mass or more, and further preferably 40 parts by mass or more, and may be 60, 80, 90, or 95 parts by mass or more, or may be 100 parts by mass.

When the polymer (A) of the present disclosure contains a constitutional unit other than the monomer (a), the constitutional unit other than the monomer (a) may be derived from any radical polymerizable monomer (hereinafter, a monomer to be a constitutional unit other than the monomer (a) by copolymerization is also referred to as “monomer (b)”).

When the polymer (A) contains a constitutional unit derived from the monomer (b), the proportion of the constitutional unit derived from the monomer (b) per 100 parts by mass of the polymer (A) is, for example, 99 parts by mass or less, preferably 80 parts by mass or less, more preferably 50 parts by mass or less, further preferably 40 parts by mass or less, even more preferably 20 parts by mass or less, and particularly preferably 10 parts by mass or less.

Examples of the monomer (b) include those other than the monomer (a) such as hydroxyl group-containing (meth)acrylate, a polyoxyalkylene group-containing monomer, alkoxyalkyl (meth)acrylate, a vinyl monomer, a cyclic compound, and the like. Only one kind of these monomers (b) may be used singly, or two or more kinds thereof may be used in combination.

Examples of the hydroxyl group-containing (meth)acrylate include hydroxyalkyl (meth)acrylate having 2 to 4 carbon atoms in a hydroxyalkyl group such as 2-hydroxyethyl acrylate and 2-hydroxyethyl methacrylate, and the like.

Examples of the polyoxyalkylene group-containing unsaturated monomer include a monomer represented by the following General Formula (6) and the like.

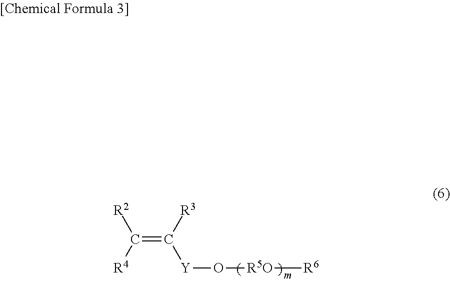

In General Formula (6), R², R³, and R⁴ each independently represent a hydrogen atom or a methyl group, R⁵ represents an alkylene group having 2 to 18 carbon atoms, R⁶ represents a hydrogen atom or a hydrocarbon group having 1 to 20 carbon atoms, Y represents an alkylene group having 1 to 5 carbon atoms, a —CO— group, or a direct bond when a R²R⁴C═CR³— group is a vinyl group, and m represents the average number of added moles of a —(R⁵O)— group and represents a number from 1 to 300. In Formula (6), when (R⁵O)ₘ consists of two or more kinds of R⁵O, the two or more kinds of R⁵O may be in any of random, block, and alternate binding forms.

In General Formula (6), R⁶ is a hydrogen atom or a hydrocarbon group having 1 to 20 carbon atoms. Among R⁶, a hydrogen atom or a hydrocarbon group having 1 to 10 carbon atoms are more preferable, a hydrogen atom or a hydrocarbon group having 1 to 3 carbon atoms are even more preferable, and a hydrogen atom or a hydrocarbon group having 1 or 2 carbon atoms are further preferable. Among the hydrocarbon groups, an alkyl group or an alkenyl group is preferable, an alkyl group having 1 to 20 carbon atoms is more preferable, an alkyl group having 1 to 10 carbon atoms is further preferable, and an alkyl group having 1 to 3 carbon atoms is still more preferable.

In General Formula (6), an oxyalkylene group represented by the formula: —R⁵O— is an oxyalkylene group having 2 to 18 carbon atoms. Examples of the oxyalkylene group include an oxyethylene group, an oxypropylene group, an oxybutylene group, an oxyisobutylene group, an oxy-1-butene group, an oxy-2-butene group, and the like. Among these oxyalkylene groups, an oxyalkylene group having 2 to 8 carbon atoms is preferable, an oxyalkylene group having 2 to 4 carbon atoms such as an oxyethylene group, an oxypropylene group, and an oxybutylene group is more preferable, and an oxyethylene group is further preferable.

In General Formula (6), m is the average number of added moles of the oxyalkylene group represented by the formula: —R⁵O—. The average number of added moles means the average number of moles of oxyalkylene groups in 1 mole of a polyoxyalkylene group-containing unsaturated monomer. The lower limit value of m is preferably 2 or more, more preferably 4 or more, and further preferably 8 or more. The upper limit value of m is preferably 100 or less and more preferably 50 or less.

X represents an alkylene group having 1 to 5 carbon atoms, a —CO— group, or a direct bond when a R²R⁴C═CR³— group is a vinyl group. Among these groups, a —CO— group is preferable.

Examples of the polyoxyalkylene group-containing unsaturated monomer include an unsaturated alcohol polyalkylene glycol adduct, a polyalkylene glycol ester-based monomer, (alkoxy) polyalkylene glycol monomaleic acid ester, and the like.

The unsaturated alcohol polyalkylene glycol adduct is a compound in which a polyalkylene glycol chain is added to an alcohol having an unsaturated group. Examples of the unsaturated alcohol polyalkylene glycol adduct include polyethylene glycol monovinyl ether, polyethylene glycol monoallyl ether, polyethylene glycol mono(2-methyl-2-propenyl) ether, polyethylene glycol mono(2-butenyl) ether, polyethylene glycol mono(3-methyl-3-butenyl) ether, polyethylene glycol mono(3-methyl-2-butenyl) ether, polyethylene glycol mono(2-methyl-3-butenyl) ether, polyethylene glycol mono(2-methyl-2-butenyl) ether, polyethylene glycol mono(1,1-dimethyl-2-propenyl) ether, polyethylene polypropylene glycol mono(3-methyl-3-butenyl) ether, methoxypolyethylene glycol mono(3-methyl-3-butenyl) ether, and the like.

The polyalkylene glycol ester-based monomer is a monomer in which an unsaturated group and a polyalkylene glycol chain are bonded via an ester bond.

As the polyalkylene glycol ester-based monomer, for example, an esterified product of alkoxypolyalkylene glycol having 1 to 300 mol of an oxyalkylene group having 2 to 18 carbon atoms added to an alcohol and (meth)acrylic acid is preferable. Among the alkoxypolyalkylene glycols, those containing an oxyethylene group as a main component are preferable. Examples of the alcohol include aliphatic alcohols having 1 to 30 carbon atoms such as methanol, ethanol, 1-propanol, 2-propanol, 1-butanol, 2-butanol, 1-pentanol, 2-pentanol, 3-pentanol, 1-hexanol, 2-hexanol, 3-hexanol, octanol, 2-ethyl-1-hexanol, nonyl alcohol, lauryl alcohol, cetyl alcohol, and stearyl alcohol, alicyclic alcohols having 3 to 30 carbon atoms such as cyclohexanol, unsaturated alcohols having 3 to 30 carbon atoms such as (meth)allyl alcohol, 3-buten-1-ol, and 3-methyl-3-buten-1-ol, and the like. Examples of the esterified product include methoxypolyethylene glycol mono(meth)acrylate, methoxy (polyethylene glycol polypropylene glycol) mono(meth)acrylate, methoxy (polyethylene glycol polybutylene glycol)mono(meth)acrylate, methoxy (polyethylene glycol polypropylene glycol polybutylene glycol)mono(meth)acrylate, and the like. Among the polyalkylene glycol ester-based monomers, for example, (alkoxy) polyalkylene glycol mono(meth)acrylate such as methoxypolyethylene glycol monomethacrylate is preferable.

Examples of the alkoxyalkyl (meth)acrylate include alkoxyalkyl (meth)acrylates, in which an alkoxy group has 1 to 4 carbon atoms and an alkyl group has 1 to 4 carbon atoms, such as methoxymethyl (meth)acrylate, methoxyethyl (meth)acrylate, methoxypropyl (meth)acrylate, ethoxymethyl (meth)acrylate, ethoxyethyl (meth)acrylate, and ethoxypropyl (meth)acrylate, and the like. These alkoxyalkyl (meth)acrylates may be used singly or in combination of two or more kinds thereof.

Examples of the vinyl monomer include (meth)acrylic acid, methyl (meth)acrylate, ethyl (meth)acrylate, propyl (meth)acrylate, n-butyl (meth)acrylate, isobutyl (meth)acrylate, tert-butyl (meth)acrylate, 2-ethylhexyl (meth)acrylate, n-lauryl (meth)acrylate, n-stearyl (meth)acrylate, diaminomethyl (meth)acrylate, diaminoethyl (meth)acrylate, dimethylamino (meth)acrylate, diethylamino (meth)acrylate, glycidyl (meth)acrylate, styrene, aziridines, 2-(meth)acryloyloxymethyl phosphorylcholine, 2-(meth)acryloyloxyethyl phosphorylcholine, tetrahydrofurfuryl (meth)acrylate, isopropylacrylamide, vinyl alcohol, vinylformamide, vinylisobutylacrylamide, (meth)acrylamide, dimethylacrylamide, vinylacetamide, N-vinylpyrrolidone, and the like.

Examples of the alkylene oxide include alkylene oxides having 2 to 4 carbon atoms such as ethylene oxide and propylene oxide, and the like.

Examples of the alkoxypolyoxyalkylene glycol include alkoxypolyoxyalkylene glycol, which has an alkoxy group having 1 to 4 carbon atoms and an oxyalkylene group having 1 to 4 carbon atoms in which the number of moles of the oxyalkylene group added is 2 to 30, such as polyethylene glycol, polypropylene glycol, methoxy polyethylene glycol, ethoxy polyethylene glycol, methoxy polypropylene glycol, ethoxy polypropylene glycol, and the like.

Examples of the cyclic compound include lactides such as L-lactide, lactones such as ε-caprolactone, trimethyl carbonate, cyclic amino acid, morpholine-2,5-dione, and the like.

When the polymer (A) consists of a polymer containing the constitutional unit derived from the monomer (a), a polymer constituting the polymer may have a configuration of a block copolymer obtained by bonding polymers of the same type or different types.

The number average molecular weight (Mn) of the polymer (A) of the present disclosure is preferably 1000 or more, more preferably 2000 or more, and further preferably 3000 or more, and may be, for example, 6000 or more, from the viewpoint of, for example, improving the enzyme resistance of the conjugate compound. The number average molecular weight (Mn) of the polymer is preferably 50000 or less, more preferably 30000 or less, and further preferably 15000 or less, from the viewpoint of extracorporeal excretion, and the like. The value of the number average molecular weight (Mn) of the polymer means a value as measured based on the method for measuring Mn of the polymers obtained in Production Example 1 to 4 in Examples described below. The number average molecular weight (Mn) of the polymer (A) of the present disclosure is preferably 1000 to 50000 and more preferably 3000 to 30000.

The polydispersity of the polymer (A) of the present disclosure (value of [polymerization average molecular weight (Mw)/number average molecular weight (Mn)]) is preferably 1.00 to 5.00, more preferably 1.00 to 3.00, further preferably 1.00 to 2.00, still more preferably 1.00 to 1.50, and even more preferably 1.00 to 1.30, from the viewpoint of uniformity of the molecular weight of the conjugate compound, and the like. The value of the polymerization average molecular weight (Mw) means a value as measured based on the above-described method for measuring Mn and measurement conditions.

### <Component (B)>

The polymer (A) of the present disclosure preferably forms a conjugate compound with the component (B) containing at least one selected from the group consisting of an amino acid, a peptide, a protein, a nucleoside, a nucleotide, and a nucleic acid.

Examples of the amino acid of the present disclosure include organic compounds containing both an amino group and a carboxyl group and salts thereof, and include not only natural amino acids (L-amino acid) but also non-natural amino acids (D-amino acid, modified amino acid, amino acid derivative, and the like).

Specific examples of the amino acid include, but are not limited to, alanine, arginine, asparagine, aspartic acid, cysteine, glutamic acid, glutamine, glycine, histidine, isoleucine, leucine, lysine, methionine, phenylalanine, serine, threonine, tyrosine, tyrosine, tryptophan, proline, and valine. Examples of other amino acids include, but are not limited to, arginosuccinic acid, citrulline, cysteinesulfinic acid, 3,4-dihydroxyphenylalanine, homocysteine, homoserine, ornithine, carnitine, selenocysteine, selenomethionine, 3-monoiodotyrosine, 3,5-diiodotyrosine, 3,5,5′-triiodothyronine, and 3,3′,5,5′-tetraiodothyronine.

The modified amino acid means an amino acid modified by addition, deletion, substitution of at least one atom, or a combination thereof (for example, N-alkylamino acid, N-acylamino acid, or N-methylamino acid). Examples of the modified amino acid include, but are not limited to, amino acid derivatives such as trimethylglycine, N-methyl-glycine, and N-methyl-alanine.

Examples of the other non-natural amino acids include, but are not limited to, D-amino acid, hydroxylysine, dehydroalanine, pyrrolidine, 2-aminoisobutyric acid, γ-aminobutyric acid, 5-hydroxytryptophan, S-adenosylmethionine, S-adenosylhomocysteine, 4-hydroxyproline, N-Cbz-protected amino acid, 2,4-diaminobutyric acid, homoarginine, norleucine, N-methylaminobutyric acid, naphthylalanine, phenylglycine, β-phenylproline, tert-leucine, 4-aminocyclohexylalanine, N-methyl-norleucine, 3,4-dehydroproline, N, N-dimethylaminoglycine, N-methylaminoglycine, 4-aminopiperidine-4-carboxylic acid, 6-aminocaproic acid, trans-4-(aminomethyl)-cyclohexanecarboxylic acid, 2-, 3-, and 4-(aminomethyl)-benzoic acid, 1-aminocyclopentanecarboxylic acid, 1-aminocyclopropanecarboxylic acid, and 2-benzyl-5-aminopentanoic acid.

The peptide of the present disclosure refers to a peptide in which an amino acid is peptide-bonded, and examples thereof include a compound in which 2 to 50 amino acids are peptide-bonded. Examples of the above-described peptide or a protein described below include peptide derivatives, peptide aptamers; antibodies such as immunoglobulins; antibody fragments, antibody derivatives, peptide-nucleic acids (PNA); hormones such as interleukin, lymphokine, and cytokine; enzymes, growth factors, and the like.

The protein of the present disclosure refers to a protein in which an amino acid is peptide-bonded, and examples thereof include a compound in which the number of amino acids to be bonded is 50 or more. Specific examples thereof include enzymes, hormones, cytokines, antibodies, and the like.

Amino acids constituting the peptide or protein of the present disclosure include not only natural amino acids but also non-natural amino acids. The primary structure of the peptide or protein of the present disclosure is a linear or cyclic structure, and may include two structures at the same time, may form a secondary structure in either a helix shape or a sheet shape, and may further form a three-dimensional structure such as a tertiary structure or a quaternary structure. In the present specification, the derivative of a peptide or a protein includes not only a peptide or a protein containing an amino acid derivative but also a protein hydrolysate obtained by partially hydrolyzing a peptide or a protein with an acid, an alkali, or an enzyme, and a derivative such as a cationized product, an acylated product, an alkyl-esterified product, or a siliconized product thereof.

The molecular weight of the protein of the present disclosure is preferably 1,000 or more, more preferably 3,000 or more, and still more preferably 5,000 or more, and is preferably 4,000,000 or less, more preferably 1,000,000 or less, and still more preferably 500,000 or less.

The molecular weight of the protein can be measured by a known method, and can be measured by a method such as SDS-PAGE or a mass spectrometer (MALDI TOF MS).

Examples of the nucleoside of the present disclosure include, but are not limited to, ribonucleosides and deoxyribonucleosides. The nucleoside may be an artificially modified nucleoside.

Examples of the nucleotide of the present disclosure include, but are not limited to, a compound in which one to three phosphates are bonded to the above-described nucleoside. The nucleotide may be an artificially modified nucleotide.

Examples of the nucleic acid of the present disclosure include, but are not limited to, DNA and RNA. Examples of the nucleic acid of the present disclosure include oligonucleotides (for example, those having 2 to 100 bases in length), gapmers, ribozymes, aptamers, artificial nucleic acids, and the like. The oligonucleotide may be siRNA, miRNA, aptamer, CpG oligo, or antisense DNA/RNA.

The nucleoside of the present disclosure is not particularly limited, but is preferably a compound in which a base such as a purine base, a pyrimidine base, nicotinamide, or dimethylisoalloxazine is bonded to a sugar.

Examples of the nucleoside of the present disclosure include, but are not limited to, adenosine, deoxyadenosine, guanosine, deoxyguanosine, 5-methyluridine, thymidine, uridine, methylpseudouridine, pseudouridine, deoxyuridine, cytidine, deoxycytidine, and the like.

The nucleotide of the present disclosure includes a natural or non-natural nucleotide. The natural nucleotide includes a deoxyribonucleotide having a base of any of adenine, guanine, cytosine, and thymine and a ribonucleotide having a base of any of adenine, guanine, cytosine, and uracil. The non-natural nucleotide includes an artificial nucleotide having a property and/or a structure similar to that of a natural nucleotide, and an artificial nucleotide containing a non-natural nucleoside or a non-natural base having a property and/or a structure similar to that of a natural nucleoside or a natural base which is a constituent element of a natural nucleotide. Examples of the non-natural nucleoside include abasic nucleoside, arabinonucleoside, 2′-deoxyuridine, α-deoxyribonucleoside, β-L-deoxyribonucleoside, and nucleosides having other sugar modifications. Examples thereof further include nucleosides having substituted pentose (2′-O-methylribose, 2′-deoxy-2′-fluororibose, 3′-O-methylribose, or 1′,2′-deoxyribose), arabinose, substituted arabinose sugar, substituted hexose, and sugar modification resulting in an alpha anomer. The non-natural nucleotide also includes a nucleotide containing an artificially constructed base analog or an artificially chemically modified base (modified base). Examples of the base analog include a 2-oxo(1H)-pyridin-3-yl group, a 5-substituted-2-oxo(1H)-pyridin-3-yl group, a 2-amino-6-(2-thiazolyl) purin-9-yl group, a 2-amino-6-(2-oxazolyl) purin-9-yl group, and the like.

Examples of the modified base include modified pyrimidine (for example, 5-hydroxycytosine, 5-fluorouracil, or 4-thiouracil), modified purine (for example, 6-methyladenine or 6-thioguanosine), other heterocyclic bases, and the like. Chemically modified nucleic acids or nucleic acid analogs such as methylphosphonate-type DNA/RNA, phosphorothioate-type DNA/RNA, phosphoramidate-type DNA/RNA, and 2′-O-methyl-type DNA/RNA can also be included. The nucleic acid analog is an artificially constructed compound having a structure and/or a property similar to that of a natural nucleic acid, and examples thereof include a peptide nucleic acid (PNA), a peptide nucleic acid having a phosphate group (PHONA), a bridged nucleic acid or locked nucleic acid (BNA/LNA), a morpholino nucleic acid, and the like.

Examples of the nucleic acid of the present disclosure include polymers in which nucleotides are linked by a phosphate diester bond, a methyl phosphonate bond, a methyl thiophosphonate bond, a phosphoromorpholidate bond, a phosphoropiperazidate bond, a phosphoroamidate bond, a phosphorothioate bond, a phosphorodithioate bond, or the like; and the like.

Examples of the DNA of the present disclosure include an embodiment in which a base selected from adenine, guanine, cytosine, and thymine is bonded to a deoxyribose ring linked via a phosphate diester bond, and these may have a substituent.

The DNA of the present disclosure may be single-stranded DNA, double-stranded DNA, or DNA-RNA hybrid, and specific examples thereof include genomic DNA, coding DNA, DNA primer, DNA probe, immunostimulatory DNA, DNA oligonucleotide, DNA polynucleotide, aDNA, plasmid, antisense DNA oligonucleotide, aptamer, decoy, viral DNA, and the like.

Examples of the RNA of the present disclosure include an embodiment in which a base selected from adenine, guanine, cytosine, and uracil is bonded to a ribose ring linked via a phosphate diester bond, and these may have a substituent.

The RNA of the present disclosure may be single-stranded RNA, double-stranded RNA, or DNA-RNA hybrid, and specific examples thereof include RNA oligonucleotide, messenger RNA (mRNA), immunostimulatory RNA, small interfering RNA (siRNA), antisense RNA, microRNA (miRNA), small nuclear RNA (snRNA), small hairpin (sh) RNA, ribosomal RNA (rRNA), transfer RNA (TRNA), messenger RNA (mRNA), viral RNA (VRNA), aptamer, ribozyme, and the like.

As the mRNA of the present disclosure, an artificial mRNA using a modified nucleic acid such as pseudouridine, N¹-methylpseudouridine, and other derivatives may be used instead of uridine in which uracil is bonded to a ribose ring.

The RNA is preferably an oligonucleotide having 15 to 50 nucleotide units, and more preferably an oligonucleotide having 20 to 30 linked nucleotide units.

As the microRNA (miRNA) of the present disclosure, miRNA in which 17 to 25 nucleotide units are linked can be used. The siRNA can include, for example, 16 to 30 nucleotide units and have a double-stranded region. In another embodiment, the nucleic acid is an immunostimulatory oligonucleotide, decoy oligonucleotide, supermir, miRNA mimic, or miRNA inhibitor. The supermir refers to single-stranded, double-stranded or partially double-stranded oligomer or polymer of RNA or deoxyribonucleic acid DNA, or both or modifications thereof, which has a nucleotide sequence that is substantially identical to miRNA and that is antisense with respect to its target. The miRNA mimics represent a class of molecules that can be used to imitate the gene silencing ability of one or more miRNAs. Thus, the term “miRNA mimic” refers to synthetic non-coding RNAs (that is, miRNA is not obtained by purification from a source of the endogenous miRNA) that are capable of entering the RNAi pathway and regulating gene expression.

The gapmer of the present disclosure means an antisense nucleic acid in which an artificial nucleic acid is arranged at both ends, and an effect of enhancing the activity of the antisense nucleic acid is expected.

The aptamer of the present disclosure is a nucleic acid molecule that binds to a target protein, and is single-stranded DNA or single-stranded RNA.

The molecular weight of the nucleic acid of the present disclosure is preferably 300 or more, more preferably 1,000 or more, and further preferably 2,000 or more, is preferably 4,000,000 or less, more preferably 3,000,000 or less, and further preferably 2,000,000 or less, and may be 1,500,000 or less.

The molecular weight of the nucleic acid can be measured by a known method, and can be measured by a method such as agarose electrophoresis, SDS-PAGE, HPLC-MS, or MALDI-TOFMS.

### <Conjugate Compound>

A mass ratio of the polymer (A) and the component (B) in the conjugate compound of the present disclosure may be appropriately set according to the types and molecular weights of the polymer (A) and the component (B), the balance between affinity and hydrophobicity, the site to be delivered, the required blood residence time, and the like, but the mass ratio thereof is not particularly limited, and from the viewpoint of efficient bonding and the like, the polymer (A)/the component (B) is preferably 1/999 to 499/1, more preferably 1/499 to 150/1, further preferably 1/499 to 99/1, and still more preferably 1/199 to 49/1.

In the conjugate compound of the present disclosure, the polymer (A) and the component (B) are preferably bonded via a divalent bonding group from the viewpoint of improving the heat resistance of the conjugate compound.

Specific examples of the divalent bonding group of the present disclosure include —S—, —S—C(═S)—, —S—C(═S)—S—, —S—C(═S)—N(—Ra)—, —S—C(═S)—O—, —S—Rb—C(═O)—O—, —S—Rb—C(═O)—N(—Ra)—, —S—Rb—O—, —S—Rb—OC(═O)—, —O—, —O—C(═O)—, —N(—Ra)—C(═O)—, and a divalent bonding group containing these. Here, Ra described above is a hydrogen atom or a hydrocarbon group having 1 to 30 carbon atoms. Rb described above is a hydrocarbon group having 1 to 30 carbon atoms. Residues obtained by excluding one hydrogen atom and one hydrocarbon group having 8 or more carbon atoms from lipids (including modified products of lipids) are also one of preferred forms of the divalent bonding group. The molecular weight of the divalent bonding group is preferably 5000 or less, more preferably 2000 or less, and further preferably 1000 or less.

From the viewpoint of bond stability, the conjugate compound of the present disclosure preferably has at least one of bonds represented by General Formulas (1) to (4), and more preferably has at least one of bonds represented by General Formulas (1) and (2). Note that, the bond of General Formula (1) is, for example, a bond generated by a combination of a thiol group-maleimide group, the bond of General Formula (2) is, for example, a bond generated by a combination of a —NH₂ group (amino group)-maleimide group, the bond of General Formula (3) is, for example, a bond generated by a combination of a —NH group (amino group)-succinimide group (N-hydroxysuccinimide (NHS) ester group; the same applies hereinafter), and the bond of General Formula (4) is, for example, a bond generated by a combination of a thiol group-succinimide group.

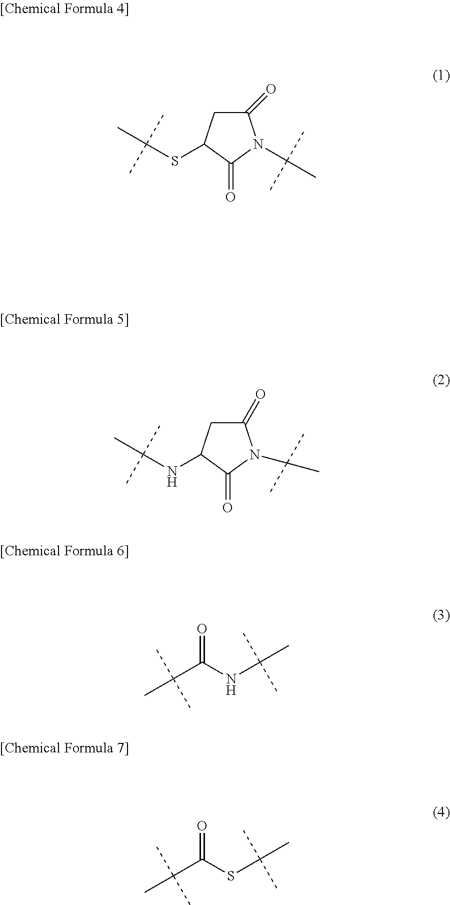

(In Formulas 1 to 4, dashed lines each indicate the attachment to the polymer (A) and the component (B).)

From the viewpoint of improving the heat resistance of the conjugate compound, the conjugate compound preferably has at least one selected from the group consisting of an azide group, a thiol group, an amino group, an alkynyl group, a maleimide group, a succinimide group, a leaving group (halogen or the like), and a disulfide group as a terminal functional group of the polymer (A) and/or the component (B), and the conjugate compound of the present disclosure preferably includes a bond generated by an azide group-alkynyl group, a thiol group-disulfide group, or a combination of a thiol group or an amino group with a maleimide group, a succinimide group, or a leaving group, more preferably includes a bond generated by a thiol group-disulfide group, or a combination of a thiol group or an amino group with a maleimide group or a succinimide group, and further preferably includes a bond generated by a combination of a thiol group-maleimide group, or an amino group-succinimide group.

The conjugate compound of the present disclosure may be any of a powder, a dispersion, a solution, and a paste, but is preferably a powder from the viewpoint of ease of storage.

### <Method for Producing Polymer (A)>

The polymer (A) of the present disclosure can be obtained by polymerizing a monomer composition containing the monomer (a) and, if necessary, the monomer (b). Examples of the method for polymerizing the monomer composition include a radical polymerization method, a living radical polymerization method represented by an atom transfer radical polymerization method, a reversible addition-fragmentation chain transfer (RAFT) polymerization method, or the like, an ionic polymerization method, a ring-opening polymerization method, a coordination polymerization method, a polycondensation method, and the like, but the present invention is not limited to these examples.

When the monomer composition is polymerized, a solvent may be used. Examples of the solvent include aromatic solvents such as benzene, toluene, and xylene; alcohol-based solvents such as methanol, ethanol, isopropanol, n-butanol, and tert-butanol; halogen atom-containing solvents such as dichloroethane and dichloromethane; ether-based solvents such as diethyl ether, propylene glycol methyl ether, dipropylene glycol methyl ether, ethyl cellosolve, and butyl cellosolve; ester-based solvents such as ethyl acetate, butyl acetate, and cellosolve acetate; ketone-based solvents such as acetone, methyl ethyl ketone, methyl isobutyl ketone, and diacetone alcohol; organic solvents such as amide-based solvents such as dimethylformamide, and water. Among them, an alcohol-based solvent is preferably used from the viewpoint of reactivity. These solvents may be used singly or in combination of two or more kinds thereof. The amount of the solvent may be appropriately determined in consideration of the polymerization conditions, the composition of the monomer composition, the concentration of the obtained polymer, and the like.

When the monomer composition is polymerized, a chain transfer agent can be used to adjust the molecular weight of the polymer and to introduce a functional group such as a hydrocarbon group or an amino group.

Examples of the chain transfer agent include hydrophilic thiol-based chain transfer agents such as alkali metal salts of thioacetate, such as sodium thioacetate and potassium thioacetate, cysteine, cysteamine, mercaptoethanol, thioglycerol, thioglycolic acid, mercaptopropionic acid, 2-mercaptopropionic acid, 3-mercaptopropionic acid, thioacetic acid, thiomalic acid, 2-mercaptoethanesulfonic acid, and sodium and potassium salts thereof; non-thiol-based chain transfer agents such as primary alcohols such as 2-aminopropan-1-ol, secondary alcohols such as isopropanol, phosphorus acid, hypophosphorous acid, and salts thereof (for example, sodium hypophosphite, potassium hypophosphite, and the like), sulfites, hydrogen sulfites, dithionous acids, metabisulfites, and salts thereof (for example, sodium sulfite, sodium hydrogen sulfite, sodium dithionate, sodium metabisulfite, potassium sulfite, potassium hydrogen sulfite, potassium dithionate, potassium metabisulfite, and the like); hydrophobic thiol-based chain transfer agents such as butanethiol, octanethiol, decanethiol, dodecanethiol, hexadecanethiol, octadecanethiol, thiocholesterol, cyclohexylmercaptan, thiophenol, octyl thioglycolate, octyl 2-mercaptopropionate, octyl 3-mercaptopropionate, mercaptopropionic acid 2-ethylhexyl ester, octanoic acid 2-mercaptoethyl ester, 1,8-dimercapto-3,6-dioxaoctane, decantrithiol, and dodecyl mercaptan; and the like. In the case of performing reversible addition-fragmentation chain transfer (RAFT) polymerization, it is necessary to use a reversible addition-fragmentation chain transfer (RAFT) agent as the chain transfer agent. Examples of such RAFT agent include 4-cyano-4-(phenylcarbonothioylthio) pentanoic acid, 2-cyano-2-propylbenzothioate, 2-cyano-2-propyldodecyltrithiocarbonate, 4-cyano-4-[(dodecylsulfanylthiocarbonyl) sulfanyl]pentanoic acid, 2-(dodecylthiocarbonothioylthio)-2-methylpropanoic acid, cyanomethyldodecylthiocarbonate, cyanomethylmethyl (phenyl) carbamothioate, bis(thiobenzoyl)disulfide, bis(dodecylsulfanylthiocarbonyl)disulfide, and the like. These chain transfer agents may be used singly or in combination of two or more kinds thereof.

The amount of the chain transfer agent may be appropriately set according to the type of the monomer contained in the monomer composition, the polymerization conditions such as polymerization temperature, the molecular weight of the target polymer, and the like, but the amount thereof is not particularly limited. However, when a polymer having a number average molecular weight of several thousand to tens of thousands is obtained, the amount of the chain transfer agent is preferably 0.1 to 20 parts by mass and more preferably 0.5 to 15 parts by mass per 100 parts by mass of the monomer.

When the monomer composition is polymerized, a polymerization initiator can be used.

Examples of the polymerization initiator include radical polymerization initiators such as azoisobutyronitrile, 2,2′-azobis(4-dimethoxy-2,4-dimethylvaleronitrile), 4,4′-azobis(4-cyanopentanoic acid), 2,2′-azobis[2-methyl-N-[1,1-bis(hydroxymethyl)-2-hydroxyethyl]propionamide], 2,2′-azobis[N-(2-hydroxyethyl)-2-methoxypropanamide], 2,2′-azobis(2-methyl-2-propenylpropanamide), 2,2′-bis(2-imidazolin-2-yl) [2,2′-azobispropane]dihydrochloride, 2,2′-azobis(propane-2-carboamidine) dihydrochloride, 2,2′-azobis[N-(2-carboxyethyl)-2-methylpropion amidine], 2,2′-azobis[2-[1-(2-hydroxyethyl)-2-imidazolin-2-yl]propane dihydrochloride, 2,2′-azobis(2,4-dimethylvaleronitrile), 2,2′-azobis(2-methylbutyronitrile), tert-butylperoxy-2-ethylhexanoate, 2,2′-azobis(isobutyronitrile), benzoyl peroxide, di-tert-butyl peroxide, cyclohexanone peroxide, and acetylacetone peroxide; living radical polymerization initiators such as bromomethylbenzene, 1-(bromomethyl)-4-methylbenzene, ethyl 2-bromoisobutyrate, hydroxyethyl 2-bromoisobutyrate, bis[2-(2′-bromoisobutyryloxy)ethyl]disulfide, 2-bromoisobutyrate 10-undecenyl, and 4-(1-bromoethyl)benzoic acid; and the like. These polymerization initiators may be used singly or in combination of two or more kinds thereof.

The amount of the polymerization initiator may be appropriately set according to the desired physical properties of the obtained polymer and the like, and usually, the amount of the polymerization initiator is preferably 0.001 to 20 parts by mass and more preferably 0.005 to 10 parts by mass per 100 parts by mass of the monomer.

The polymerization conditions for polymerizing the monomer composition may be appropriately set according to the polymerization method, and are not particularly limited. The polymerization temperature is preferably room temperature to 200° C. and more preferably 40 to 140° C. The atmosphere for polymerizing the monomer composition is preferably an inert gas such as nitrogen gas or argon gas. The reaction time may be appropriately set so that the polymerization reaction of the monomers is completed.

As described above, preferably by polymerizing the monomer composition as described above, a polymer can be obtained. Here, the obtained polymer may be used as it is as the polymer (A), but the polymer preferably has a functional group at the terminal thereof when forming a conjugate with the component (B). Since the polymer has a functional group at the terminal, the polymer can be easily linked to the component (B) via the functional group.

As the functional group that the polymer (A) of the present disclosure can have, an anionic functional group, a cationic functional group, a nonionic functional group, and an amphoteric functional group are preferable. The functional group is preferably a reactive functional group. Examples of the suitable reactive functional group include a —SH group, a group represented by the formula:—COOM (M represents a hydrogen atom or an alkali metal atom), a hydroxyl group, an allyl group, an epoxy group, an aldehyde group, a —NH₂ group (amino group), a CONH— group, and the like. Examples of the M include alkali metal atoms such as a sodium atom and a potassium atom. Among them, from the viewpoint of ease of formation of a conjugate with the component (B) and ease of formation of a bond with a linker when a conjugate having a linker between the polymer (A) and the component (B) is synthesized, a suitable reactive functional group is a —NH₂ group (amino group). When the polymer has a functional group at the terminal, the number of functional groups is not particularly limited, and is preferably 1 to 6, more preferably 1 to 4, and further preferably 1 to 2.

In order to introduce a functional group into the terminal of the polymer (A) of the present disclosure, a functional group-containing compound for introducing a functional group into the polymer can be used. Examples of the functional group-containing compound for introducing a functional group into the terminal of the polymer include thiol-based chain transfer agents such as alkali metal salts of thioacetate, such as sodium thioacetate and potassium thioacetate, cysteine, cysteamine, mercaptoethanol, thioglycerol, thioglycolic acid, mercaptoproponic acid, 2-mercaptoproponic acid, 3-mercaptoproponic acid, thioacetic acid, thiomalic acid, 2-mercaptoethanesulfonic acid, sodium and potassium salts thereof, and 16-amino-1-hexadecanethiol hydroxychloride; polymerization initiators into which the functional group is introduced, such as 4,4′-azobis(4-cyanopentanoic acid), 2,2′-azobis[2-methyl-N-[1,1-bis(hydroxymethyl)-2-hydroxyethyl]propionamide], 2,2′-azobis[N-(2-hydroxyethyl)-2-methoxypropanamide], 2,2′-azobis(2-methyl-2-propenylpropanamide), 2,2′-bis(2-imidazolin-2-yl) [2,2′-azobispropane]dihydrochloride, 2,2′-azobis(propane-2-carboamidine) dihydrochloride, 2,2′-azobis[N-(2-carboxyethyl)-2-methylpropion amidin], 2,2′-azobis[2-[1-(2-hydroxyethyl)-2-imidazolin-2-yl]propane dihydrochloride, cyclohexanone peroxide, and acetylacetone peroxide; and the like. These functional group-containing compounds may be used singly or in combination of two or more kinds thereof. The above-described functional group-containing compounds include those corresponding to the above-described chain transfer agents and polymerization initiators, but the functional group-containing compounds corresponding to the chain transfer agents and the polymerization initiators may be used for the purpose of only one of the chain transfer agent or the polymerization initiator, and the functional group-containing compound, or may be used for both purposes.

In the case of using a living polymerization initiator as the polymerization initiator, a functional group may be introduced into the terminal of the polymer by reacting a functional group-containing compound with a halogen atom present at the terminal of the polymer prepared by using the living polymerization initiator. Examples of the functional group-containing compound capable of reacting with such a halogen atom to introduce a functional group into the terminal of the polymer include amine compounds such as ethylenediamine and propyldiamine, dithiol compounds such as ethanedithiol, propanedithiol, and hexadecanedithiol, thiol compounds such as, including allyl mercaptone, cysteine, cysteamine, mercaptoethanol, thioglycerol, thioglycolic acid, mercaptopropionic acid, 2-mercaptopropionic acid, 3-mercaptopropionic acid, thioacetic acid, thiomalic acid, 2-mercaptoethanesulfonic acid, and sodium and potassium salts thereof, and the like.

The amount of the functional group-containing compound for introducing a functional group into the terminal of the polymer (A) of the present disclosure may be appropriately set according to the type of the monomer (constitutional unit) constituting the polymer, the polymerization conditions such as polymerization temperature, the molecular weight of the target polymer, and the like, but the amount thereof is not particularly limited. When a polymer having a number average molecular weight of several thousand to tens of thousands is obtained, the amount of the chain transfer agent is preferably 0.1 to 20 parts by mass and more preferably 0.5 to 15 parts by mass per 100 parts by mass of the monomer.

Examples of the method for introducing a functional group into the terminal of the polymer (A) include:
(1) a method for obtaining a polymer by polymerizing a monomer composition in the presence of a polymerization initiator into which the functional group is introduced as a polymerization initiator,
(2) a method for obtaining a polymer by polymerizing a monomer composition in the presence of a chain transfer agent into which the functional group is introduced as a chain transfer agent,
(3) a method for reacting a halogen atom present at a terminal of a polymer with a functional group-containing compound, and the like, but the present invention is not limited to such examples.

When the polymer (A) of the present disclosure forms a conjugate compound with the component (B), the polymer (A) preferably has a functional group derived from a maleimide structure or a succinimide structure (also referred to as a maleimide group or a succinimide group) at the terminal, in addition to or instead of the functional group described above. The structure can serve as a linker structure of the polymer (A) and the component (B). By having the functional group, chemical selective bonding with the component (B) becomes easier. For example, since the maleimide structure reacts with a thiol group and the succinimide structure reacts with an amino group, the bondability to a protein of an antibody or the like or a compound having a thiol group introduced into the terminal is improved.

In order to introduce a functional group derived from a maleimide structure or a succinimide structure into the terminal of the polymer, examples of such a method include:
(1) a method for obtaining a polymer by polymerizing a monomer composition in the presence of a chain transfer agent having a maleimide group or a succinimide group or an RAFT agent such as 4-cyano-4-(phenylcarbonothioylthio) pentanoic acid N-succinimidyl ester,
(2) a method for reacting a functional group (for example, amino group) present at a terminal of a polymer with a succinimide group (N-hydroxysuccinimide (NHS) ester group) in a maleimide group-containing compound (for example, N-succinimidyl 4-maleimidobutyrate), and the like, but the present invention is not limited to these examples.

### <Method for Producing Conjugate Compound>

The conjugate compound of the present disclosure is preferably obtained by a method including a step of reacting a polymer (A) having a constitutional unit derived from a monomer (a) having two or more hydroxyl groups and having 2 to 10 carbon atoms constituting a side chain among carbon atoms of the constitutional unit with a component (B) containing any one selected from the group consisting of an amino acid, a polypeptide, a protein, a nucleoside, a nucleotide, and a nucleic acid at a mass ratio, the polymer (A)/the component (B), of preferably 1/999 to 499/1.

The monomer and physical properties used for the polymer (A) and the compound used for the component (B) are as described above.

The polymer (A) of the present disclosure preferably has a functional group at the terminal, and more preferably has a maleimide structure or a succinimide structure.

The component (B) of the present disclosure preferably has a functional group in the molecule when a conjugate compound is formed. As such a functional group, a functional group uniquely possessed by the component (B) may be used as in a case where the component (B) has an amino group or a thiol group (for example, a cysteine residue in a protein) in the molecule as in an amino acid or a protein, or the functional group may be introduced into an amino acid, a polypeptide, a protein, a nucleoside, a nucleotide, or a nucleic acid. A method for introducing a functional group into an amino acid, a polypeptide, a protein, a nucleoside, a nucleotide, or a nucleic acid is known. For example, in the introduction of a thiol group into a nucleic acid, a thiol group may be introduced (generated) by introducing a disulfide bond into a nucleic acid, and then reducing the introduced disulfide bond using a reducing agent such as dithiothreitol, 2-mercaptoethanol, or tris(2-carboxyethyl) phosphine hydrochloride (TCEP). Furthermore, for example, a thiol group may be introduced (generated) by reducing a disulfide bond in a protein using a reducing agent such as dithiothreitol, 2-mercaptoethanol, tris(2-carboxyethyl) phosphine hydrochloride (TCEP).

Examples of the functional group at the terminal of the component (B) include an azide group, a thiol group, an amino group, an alkynyl group, a maleimide group, a succinimide group, a leaving group (halogen or the like), a disulfide group, and the like, and from the viewpoint of universality in biologically-related molecules and easiness of availability and synthesis of materials, a thiol group, an amino group, and a disulfide group are more preferable, and a thiol group and an amino group are still more preferable.

In a preferred embodiment, the component (B) is at least one selected from the group consisting of an amino acid, a polypeptide, a protein, a protein into which a thiol group is introduced, a nucleoside into which a thiol group is introduced, a nucleotide into which a thiol group is introduced, and a nucleic acid into which a thiol group is introduced.

The conjugate compound of the present disclosure is preferably obtained by reacting a terminal functional group of the polymer (A) with at least one selected from the group consisting of an azide group, a thiol group, an amino group, an alkynyl group, a maleimide group, a succinimide group, and a leaving group of the component (B). In a preferred embodiment of the production method, the polymer (A) has a maleimide structure or a succinimide structure at a terminal, the component (B) has at least one selected from the group consisting of an azide group, a thiol group, an amino group, an alkynyl group, a maleimide group, a succinimide group, and a leaving group, and the maleimide structure or the succinimide structure is reacted with the at least one selected from the group consisting of an azide group, a thiol group, an amino group, an alkynyl group, a maleimide group, a succinimide group, and a leaving group. In another preferred embodiment of the production method, (1) the polymer (A) has a maleimide structure at a terminal, the component (B) has a thiol group, and the maleimide structure is reacted with the thiol group, or (2) the polymer (A) has a succinimide structure at a terminal, the component (B) has an amino group, and the succinimide structure is reacted with the amino group.

The above-described reaction may be performed in a buffer solution. The reaction is preferably performed at around room temperature in order to maintain the activity of the component (B), and may be performed, for example, at 5 to 40° C. or 20 to 30° C. The reaction time is appropriately set in consideration of the reactivity of the polymer (A) and the component (B), and the like, and is, for example, 30 minutes to 10 days.

### <Use Application of Conjugate Compound>

Since the conjugate compound of the present disclosure has high biocompatibility, the conjugate compound can be suitably used for medical use applications. That is, according to another embodiment of the present invention, there is provided a pharmaceutical containing the conjugate compound according to the present disclosure. The pharmaceutical may contain the conjugate compound according to the present disclosure, or may be a composition further containing other components. Examples of the other components include water, saline, a pharmaceutically acceptable organic solvent, collagen, polyvinyl alcohol, polyvinylpyrrolidone, a carboxyvinyl polymer, a sodium carboxymethyl cellulose salt, sodium polyacrylate, sodium alginate, water-soluble dextran, sodium carboxymethyl starch, pectin, methyl cellulose, ethyl cellulose, xanthan gum, gum arabic, casein, gelatin, agar, diglycerin, propylene glycol, polyethylene glycol, petrolatum, paraffin, stearyl alcohol, stearic acid, human serum albumin, mannitol, sorbitol, lactose, a phosphate buffered saline, a biodegradable polymer, a serum-free medium, surfactant acceptable as a pharmaceutical additive, a physiological pH buffer acceptable in vivo, and the like. These additives may be used singly or in combination of two or more kinds thereof.

The conjugate compound of the present disclosure or the composition containing the conjugate compound is preferably used as a liquid preparation, a solid preparation, or a gel preparation.

The liquid preparation of the present disclosure preferably contains water, saline, a phosphate buffered saline, a citrate-phosphate buffer, and the like, in addition to the conjugate compound of the present disclosure.

The solid preparation of the present disclosure preferably contains an excipient such as mannitol, xylitol, maltodextrin, sodium carboxymethylcellulose, polyethylene glycol, agar, or lactose, in addition to the conjugate compound of the present disclosure.

The gel preparation of the present disclosure preferably contains a neutralized anionic polymer such as polyacrylic acid, carboxypolymethylene and carboxymethylcellulose, Pemulen, a polymer emulsifier, a thickener such as polycarbophil, a lower alcohol such as ethanol or isopropanol, and water, in addition to the conjugate compound of the present disclosure.

The conjugate compound of the present disclosure or the composition containing the conjugate compound can be suitably used as a pharmaceutical additive when a medicinal ingredient is not contained in its configuration. Examples of the pharmaceutical additive include a carrier for holding a medicine or the like; and the like. As a method for holding a medicine or the like with the conjugate compound according to the present invention or a medical resin composition, it is preferable to use a component in which the component (B) of the present disclosure itself is an active pharmaceutical ingredient.

The conjugate compound or a pharmaceutical composition containing the conjugate compound can be utilized for either in vitro or in vivo testing. As a method of administering the conjugate compound or the like to a living body, parenteral administration, that is, intraarticular administration, intravenous administration, intraperitoneal administration, subcutaneous administration, or intramuscular administration is preferably employed. The intravenous or intraperitoneal administration of the (pharmaceutical) composition can be performed by bolus injection.

Since the conjugate compound of the present disclosure or a medicine containing the conjugate compound suppresses complement activation, the conjugate compound of the present disclosure or the medicine containing the conjugate compound is expected to be a pharmaceutical that does not cause an unintended adverse immune reaction and has favorable blood retention.

### EXAMPLES

### [Measurement of Average Molecular Weight of Polymer]

The number average molecular weight of polymers produced in Production Examples 1 to 4 described below was measured by gel permeation chromatography (GPC). At this time, the measurement conditions were as follows.

### [Measurement Conditions of Number Average Molecular Weight of Polymer]

Measuring equipment: manufactured by Tosoh Corporation, product number: HLC-8320GPC
Molecular weight column: manufactured by Tosoh Corporation, product number: two columns of TSKgel SuperAWM-H and SuperAW2500 are connected in series
Eluent: Dimethylformamide added with 10 mmol/L lithium bromide
Standard substance for calibration curve: polystyrene
Preparation of solution for measurement: A solution in which a polymer is dissolved in dimethylformamide to have a polymer concentration of 0.2 mass % is prepared, and a filtrate after filtering the solution with a filter is used.

### Production Example of Terminal Functional Group-Introduced Compound (Polymer)

### Production Example 1

In a side arm test tube, 1.0 g of glycerol monoacrylate, 0.046 g of 16-amino-1-hexadecanethiol hydroxychloride, 0.015 g of 2,2′-azobis(isobutyronitrile), 3.2 mL of ethanol, and 0.8 mL of butanol were charged. Next, the inside of the tube was purged with nitrogen, and the mixture was stirred at 80° C. for 2 hours. The supernatant of the obtained reaction liquid was removed to obtain polyglycerol monoacrylate containing an amino group (ammonium group) at the terminal. The number average molecular weight of the obtained compound (polymer) was 8000.

Thereafter, in the side arm test tube, 0.640 g of the polyglycerol monoacrylate containing an amino group (ammonium group) at the terminal obtained above, 0.643 g of N-succinimidyl 4-maleimidobutyrate, 9 mL of dimethylsulfoxide, and 1 mL of a triethylamine acetate solution (2 mol/L, pH 7.0) were charged. Next, the inside of the tube was purged with nitrogen, and the mixture was stirred at room temperature for 23 hours. The obtained reaction liquid was diluted 5 times with water, and then subjected to gel filtration purification (PD-MidiTrap™ G-25 manufactured by Cytiva) to obtain polyglycerol monoacrylate having a maleimide structure introduced into the terminal (Polymer 1). The number average molecular weight and the polydispersity Mw/Mn of the obtained compound (Polymer 1) were 9200 and 1.96, respectively.

### Production Example 2

In a Schlenk flask with a three-way cock, 1.0 g of glycerol monomethacrylate, 0.022 g of N-hydroxysuccinimidyl 4-cyano-4-(phenylcarbonothioylthio) pentanoate, 0.016 g of 2,2′-azobis(2,4-dimethylvaleronitrile), 0.8 g of ethanol, and 0.2 g of n-butanol were charged. Next, the inside of the tube was purged with nitrogen, and the mixture was stirred at 50° C. for 30 minutes. The obtained reaction liquid was added dropwise to diethyl ether for purification to obtain polyglycerol monomethacrylate having a succinimide group introduced into the terminal (Polymer 2). The number average molecular weight and the polydispersity Mw/Mn of the obtained compound (Polymer 2) were 25000 and 1.73, respectively.

### Production Example 3

In a side arm test tube, 1.0 g of glycerol monomethacrylate, 0.046 g of 16-amino-1-hexadecanethiol hydroxychloride, 0.015 g of 2,2′-azobis(isobutyronitrile), 3.2 mL of ethanol, and 0.8 mL of butanol were charged. Next, the inside of the tube was purged with nitrogen, and the mixture was stirred at 80° C. for 2 hours. The supernatant of the obtained reaction liquid was removed to obtain polyglycerol monomethacrylate containing an amino group (ammonium group) at the terminal. The number average molecular weight of the obtained compound (polymer) was 8500.

Thereafter, in the side arm test tube, 0.630 g of the polyglycerol monomethacrylate containing an amino group (ammonium group) at the terminal obtained above, 0.643 g of N-succinimidyl 4-maleimidobutyrate, 9 mL of dimethylsulfoxide, and 1 mL of a triethylamine acetate solution (2 mol/L, pH 7.0) were charged. Next, the inside of the tube was purged with nitrogen, and the mixture was stirred at room temperature for 23 hours. The obtained reaction liquid was diluted 5 times with water, and then subjected to gel filtration purification (PD-MidiTrap™ G-25 manufactured by Cytiva) to obtain polyglycerol monomethacrylate having a maleimide structure introduced into the terminal (Polymer 3). The number average molecular weight and the polydispersity Mw/Mn of the obtained compound (Polymer 3) were 10500 and 1.98, respectively.

### Production Example 4

In a Schlenk flask with a three-way cock, 1.0 g of glycerol monoacrylate, 0.022 g of N-hydroxysuccinimidyl 4-cyano-4-(phenylcarbonothioylthio) pentanoate, 0.016 g of 2,2′-azobis(2,4-dimethylvaleronitrile), 0.8 g of ethanol, and 0.2 g of n-butanol were charged. Next, the inside of the tube was purged with nitrogen, and the mixture was stirred at 50° C. for 30 minutes. The obtained reaction liquid was added dropwise to diethyl ether for purification and dried under reduced pressure to obtain polyglycerol monoacrylate having a succinimide group introduced into the terminal (Polymer 4). The number average molecular weight and the polydispersity Mw/Mn of the obtained compound (Polymer 4) were 22000 and 1.89, respectively.

### Example 1: Preparation of Conjugate of Polymer and Protein

Conjugate buffer A (pH 7.2) was prepared by dissolving 0.52 g of sodium dihydrogen phosphate dihydrate, 2.41 g of disodium hydrogen phosphate dodecahydrate, 8.77 g of sodium chloride, and 3.73 g of EDTA·2Na in 1 L of ultrapure water. In 1 mL of this Conjugate buffer A, 41.5 mg of bovine serum albumin (FUJIFILM Wako Pure Chemical Corporation) and 66 mg of Polymer 1 prepared in Production Example 1 were each dissolved, after the dissolution, 0.1 mL of each solution was put in a test tube and mixed, and the mixture was incubated at 25° C. for 1 hour to obtain a reaction product. After incubation, the reaction product was concentrated using an ultrafiltration unit (Amicon Ultra 50K device, Merck Corporation), then diluted with PBS, and concentrated again with the ultrafiltration unit. This operation was repeated three times, and unreacted Polymer 1 was removed to obtain Conjugate 1 (PGLMA-Mal-BSA) in which the maleimide group of Polymer 1 was bonded to the thiol group of BSA.

Also for each of Polymer 2, 3, and 4 prepared in Production Examples 2, 3, and 4, a conjugate was prepared by the same method as described above to prepare Conjugate 2 (PGLMMA-Suc-BSA) in which the succinimide group of Polymer 2 was bonded to the amino group of BSA, Conjugate 3 (PGLMMA-Mal-BSA) in which the maleimide group of Polymer 3 was bonded to the thiol group of BSA, and Conjugate 4 (PGLMA-Suc-BSA) in which the succinimide group of Polymer 4 was bonded to the amino group of BSA.

### [Polyacrylamide Electrophoresis (SDS-PAGE)]

Conjugate 1 (PGLMA-Mal-BSA) subjected to ultrafiltration treatment was diluted with an appropriate amount of ultrapure water. To 20 μL of the diluted solution, 20 μL of a sample processing buffer (sample buffer solution (containing 3-mercapto-1,2-propanediol) (×2), FUJIFILM Wako Pure Chemical Corporation) was mixed, and heated at 95° C. for 10 minutes. The heated sample was applied to a precast gel (ehr-T10L e-PAGEL HR 10%, ATTO Corporation) and subjected to SDS-PAGE. As a molecular weight marker, Multicolor Protein Ladder (10-315 kDa, NIPPON GENE CO., LTD.) was used. After electrophoresis, the gel was dyed with Quick CBB Plus (FUJIFILM Wako Pure Chemical Corporation), decolored as appropriate, and photographed with a gel photographing apparatus (GEL Doc Go, Bio-Rad Laboratories, Inc.).

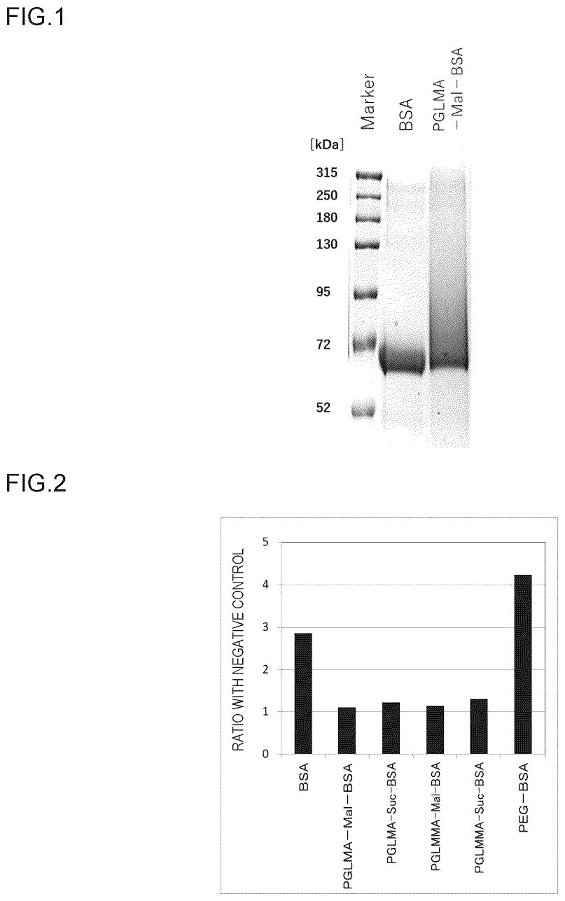

As a result, as shown in FIG. 1, it was confirmed that one band was observed in BSA alone, whereas the band was widened toward the upper part of the gel in PGLMA-Mal-BSA, and the terminal functional group-introduced PGLMA was bonded to BSA to form a conjugate.

### [Evaluation of Complement Activation (1)]

Using a microspectrophotometer (NanoDrop ND-1000, Thermo Fisher Scientific Inc.), Conjugates 1 to 4 subjected to ultrafiltration treatment were each diluted with PBS so that the value at a wavelength of 280 nm was 100. On the other hand, a conjugate (PEG-BSA) prepared according to the description in the section of [Preparation of conjugate with protein] except that maleimide PEG (SUNBRIGHT ME-100MA, NOF CORPORATION) was used instead of Polymer 1, and 100 mg of BSA were each dissolved in 1 mL of PBS to prepare a control group. Each solution (40 μL) and human serum (purchased from Tennessee Blood Service) (160 μL) were mixed and incubated at 37° C. for 1 hour. To 50 μL of the mixed solution after incubation, 5 μL of a 50 mM EDTA solution was added to stop the reaction. For each mixed solution, complement activation was evaluated using an enzyme immunoassay kit (MicroVue™ SC5b-9 Plus EIA, Quidel Corporation). The complement concentration was measured according to the manufacturer's protocol, and the measurement was performed on a plate reader (SH-9000, CORONA ELECTRIC Co., Ltd.). A reaction liquid obtained by reacting PBS with human serum was used as a negative control, and a ratio of the measured value of the negative control and a measured value in each reaction liquid was calculated by the following formula.

(Ratio with negative control)=(Measured value in each sample)÷(Measured value of negative control)

As a result, as shown in FIG. 2, it was found that complement activation was suppressed in Conjugates 1 to 4 as compared with BSA, whereas strong complement activation occurred in PEG-BSA as compared with BSA alone.

[Analysis of reaction product of Polymer 2 and protein]

A conjugate (Conjugate 2) of Polymer 2 and a protein prepared in Example 1 was analyzed by size exclusion chromatography. At this time, the measurement conditions were as follows.

### [Conditions for HPLC Analysis of Reaction Product of Polymer 2 and Protein]

Measuring equipment: HPLC (column oven: CTO-20AC, pump: LC-20AD, degasser: DGU-20A, autosampler: SIL-20AC HT, detector: ELSD-LTII) manufactured by SHIMADZU CORPORATION
Analytical column: size exclusion chromatography column (TSKgel G3000SWXL, Tosoh Corporation)
Eluent: 20 mM acetic acid-ammonium acetate (pH 4.00)
Preparation of solution for measurement: A solution in which only a conjugate or BSA is dissolved in an eluent to have a solute concentration of 0.1 mass % is prepared, and a filtrate after filtering the solution with a filter is used.

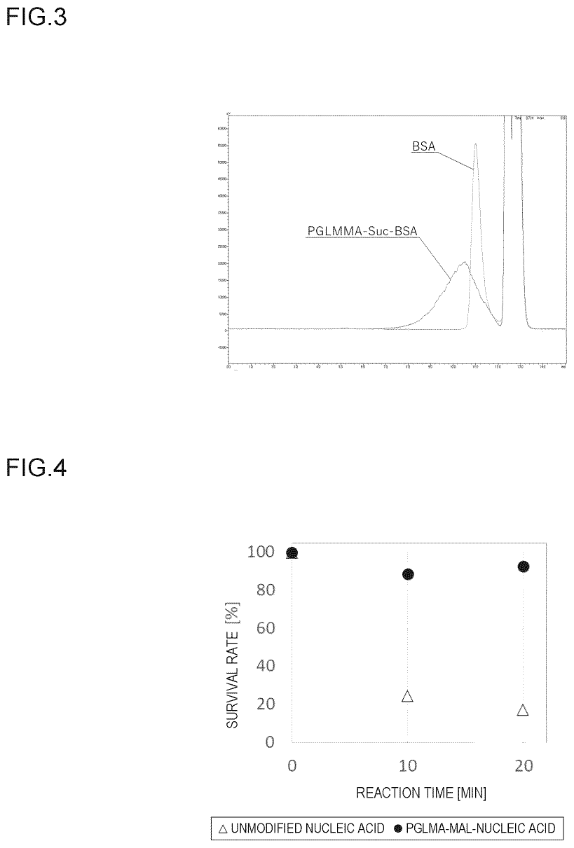

As a result of analyzing Conjugate 2 (PGLMMA-Suc-BSA) according to the above-described analysis conditions, as shown in FIG. 3, the peak of PGLMMA-Suc-BSA appeared earlier than the peak of BSA alone. This indicates that BSA was polymerized, and it became clear that a conjugate was formed.

### Example 2: Preparation of Conjugate of Polymer and Oligonucleic Acid

As the nucleic acid, an oligonucleic acid (obtained by requesting synthesis from Hokkaido System Science Co., Ltd.) having a base sequence of (5′-d (TAGCACCATGGTTT)-3′) with reference to a known paper (P. S. Eder, R. J. DeVine, J. M. Dagle, J. A. Walder (1991) Antisense Research and Development, 1 (2), 141-51), and an oligonucleic acid (obtained by requesting synthesis from Hokkaido System Science Co., Ltd.) in which 3-(propyldisulfanyl) propan-1-ol was introduced into the terminal 3′ thereof were used. First, the nucleic acid of terminal 3-(propyldisulfanyl) propan-1-ol was dissolved in ultrapure water, 160 μL (16.0 μmol) of a 0.1 M DTT solution (solution obtained by dissolving 15.5 mg of (±)-dithiothreitol) in 1.0 mL of ultrapure water) was added to 320 μL (14.3 nmol) of an oligonucleic acid solution prepared to a concentration of 44.8 μM, and the mixture was allowed to stand still at room temperature (about 25° C.) for 30 minutes to obtain a reaction liquid containing an oligonucleic acid having a thiol group at the terminal. About 1 mL of ethyl acetate was added to the prepared reaction liquid containing a terminal thiol oligonucleic acid, and then shaken well to remove the ethyl acetate layer, thereby performing washing. This washing operation was repeated 5 times to obtain a terminal thiol oligonucleic acid solution. To the obtained terminal thiol oligonucleic acid solution, a solution of Polymer 1 in which 7.8 mg of the polymer prepared in Production Example 1 was dissolved in 320 μL of ultrapure water was added and mixed (Polymer 1:oligonucleic acid=130:1 (mass ratio)), and then the mixture was allowed to stand still at room temperature (about 25° C.) for 7 days to obtain a reaction product. This reaction product was purified under the conditions described in the section of [Purification of reaction product of Polymer 1 and oligonucleic acid] to obtain a solution containing Conjugate 5 (PGLMA-Mal-nucleic acid) in which the maleimide group of Polymer 1 was bonded to the thiol group of the nucleic acid. The obtained solution containing PGLMA-Mal-nucleic acid was desalted using Illustra™ Nap™-10 Columns Sephadex™ G-25 DNA Grade (Cytiva), then freeze-dried, and dissolved in 300 μL of ultrapure water to obtain a PGLMA-Mal-nucleic acid solution (23.5 μM).

Also for Polymer 3 prepared in Production Example 3, a conjugate with an oligonucleic acid was prepared by the same method as in the case of Polymer 1 described above, except that the weight and the liquid amount were changed to 1/10, and a reaction product was obtained. Each obtained reaction product was purified using an ion exchange spin column (Vivapure (registered trademark) Q Mini H, SARTORIUS). The purification method was in accordance with the manufacturer's protocol, and 40 μL of each of ultrapure aqueous solutions containing Conjugate 6 (PGLMMA-Mal-nucleic acid) was obtained.

### [Purification of Reaction Product of Polymer 1 and Oligonucleic Acid]

The reaction product produced in the reaction between Polymer 1 and an oligonucleic acid described in Example 2 was purified by ion exchange chromatography. At this time, the purification conditions were as follows.

### [Purification Conditions of Reaction Product of Polymer 1 and Oligonucleic Acid]

Measuring equipment: AKTA avant 150 manufactured by Cytiva
Purification column: BioPro SmartSep Q20 250× 6.0 mm I.D. manufactured by Cytiva
Column temperature: room temperature (about 25° C.)
Eluent: (Eluent A) 20 mM phosphate buffer (pH 8.0), 15% acetonitrile, (Eluent B) 20 mM phosphate buffer (pH 8.0), 0.75 M sodium bromide, 15% acetonitrile
Elution conditions: Equilibration (100% Eluent A, 14 mL)→sample load (1 mL)→column washing (100% Eluent A, 7 mL)→elution (after flowing 7 mL of 100% Eluent A, flowing 21 mL while increasing the concentration of Eluent B from 15% to 75%)
Flow rate: 150 cm/h
Detection method: UV (wavelength: 260 nm)

### [Quantitative Analysis of Conjugate of Polymer 1 and Oligonucleic Acid and Unmodified Oligonucleic Acid]

Quantitative analysis of PGLMA-Mal-nucleic acid and an unmodified nucleic acid in a nucleic acid degradation test described below was performed by ion exchange chromatography. At this time, the analysis conditions were as follows.

### [Analysis Conditions of Conjugate of Polymer 1 and Oligonucleic Acid]

Measuring equipment: 1260 Infinity II manufactured by Agilent Technologies, Inc.
Analytical column: BioPro IEX SmartSep Q20 Ion Exchange Screening Kit manufactured by YMC CO., LTD.
Column temperature: room temperature (about 25° C.)
Eluent: (Eluent A) 20 mM phosphate buffer (pH 8.0), 15% acetonitrile, (Eluent B) 20 mM phosphate buffer (pH 8.0), 0.75 M sodium bromide, 15% acetonitrile
Eluent gradient: 0-4.00 min (0-15% Eluent B), 4.00-10.00 min (15-100% Eluent B), 10.00-12.00 min (100% Eluent B), 12.00-12.10 min (100-0% Eluent B), 12.10-15.00 min (0% Eluent B)
Flow rate: 0.5 mL/min
Detection method: UV (wavelength: 260 nm)
Preparation of solution for measurement: The reaction liquid after each reaction time is collected, and heated at 80° C. for 15 minutes to deactivate Exonuclease I. A solution obtained by adding 85 μL of ultrapure water to 15 μL of the reaction liquid after deactivation is used as a solution for measurement.

### [Analysis Conditions of Unmodified Oligonucleic Acid]

Measuring equipment: ACQUITY UPLC H-Class manufactured by Cytiva
Analytical column: TSKgel Super-Octyl 2.0 mm I.D.×10 cm manufactured by Tosoh Corporation
Column temperature: 60° C.
Eluent: (Eluent A) 100 mM triethylamine acetate buffer (pH 7.0), (Eluent B) purified water:acetonitrile=1:1
Eluent gradient: 0-10.00 min (2-30% Eluent B), 10.00-10.50 min (30-80% Eluent B), 10.50-15.00 min (80% Eluent B), 15.00-15.50 min (80-2% Eluent B), 15.50-20.00 min (2% Eluent B)
Flow rate: 0.2 mL/min
Detection method: UV (wavelength: 270 nm)
Preparation of solution for measurement: A solution subjected to the same treatment as in the section of [Analysis conditions of conjugate of Polymer 1 and oligonucleic acid] is used as a solution for measurement.

### [Nucleic Acid Degradation Test]

To a test tube, 42.5 μL (1.00 nmol) of the PGLMA-Mal-nucleic acid solution obtained in the section of [Preparation of conjugate of Polymer 1 and oligonucleic acid], 5 μL of 10× buffer solution (670 mM Glycine-KOH (pH 9.5), 10 mM DTT, 67 mM MgCl2), 2.5 μL of ultrapure water, and 0.5 μL of Exonuclease I (Takara Bio Inc.) (0.05 Unit/L as the final concentration of Exonuclease I in the mixed solution) were added and mixed, and the mixture was allowed to stand still at room temperature (about 25° C.). As a control group, to a test tube, 10 μL (1.00 nmol) of a solution prepared by dissolving the unmodified nucleic acid (5′-d (TAGCACCATGGTTT)-3′) used in the section of [Preparation of conjugate of Polymer 1 and nucleic acid] in ultrapure water so as to be 100 μM, 5 μL of 10× buffer solution, 34.5 μL of ultrapure water, and 0.5 μL of Exonuclease I (Takara Bio Inc.) (0.05 Unit/μL as the final concentration of Exonuclease I in the mixed solution) were added and mixed, and the mixture was allowed to stand still at room temperature (about 25° C.). After 0, 10, and 20 minutes from the start of the reaction, the reaction liquid was collected and analyzed under the analysis conditions in the section of [Analysis conditions of conjugate of Polymer 1 and oligonucleic acid] or the section of [Analysis conditions of unmodified oligonucleic acid], and a residual rate of the nucleic acid was calculated from the following formula.

$$( Residual rate ) [ \% ] = ( Area \% of PGLMA - Mal - nucleic acid or unmodified nucleic acid after each reaction time ) \div ( Area \% of PGLMA - Mal - nucleic acid or unmodified nucleic acid at the start of reaction ) \times 100$$

As a result, as shown in FIG. 4, the degradation of the nucleic acid was observed over time in the unmodified nucleic acid, but remarkable degradation of the nucleic acid was not observed in PGLMA-Mal-nucleic acid.

### [Evaluation of Complement Activation (2)]

Using a microspectrophotometer (NanoDrop ND-1000, Thermo Fisher Scientific Inc.), Conjugates 5 and 6 thus obtained were each diluted with PBS so that the value at a wavelength of 260 nm was 100. On the other hand, a conjugate (PEG-nucleic acid) prepared according to the description in the section of [Preparation of conjugate of polymer and oligonucleic acid] except that maleimide PEG (SUNBRIGHT ME-100MA, NOF CORPORATION) was used instead of Polymer 3, and the oligonucleic acid (base sequence (5′-d (TAGCACCATGGTTT)-3′)) described in Example 2 were each dissolved in 20 μL of PBS so that the value at a wavelength of 260 nm was 100 by the method described above to prepare a control group. Each solution (4 μL) and human serum (purchased from Tennessee Blood Service) (16 μL) were mixed and incubated at 37° C. for 1 hour. To 15 μL of the mixed solution after incubation, 1.5 μL of a 50 mM EDTA solution was added to stop the reaction. For each mixed solution, complement activation was evaluated using an enzyme immunoassay kit (MicroVue™ SC5b-9 Plus EIA, Quidel Corporation). The complement concentration was measured according to the manufacturer's protocol, and the measurement was performed on a plate reader (SH-9000, CORONA ELECTRIC Co., Ltd.). A reaction liquid obtained by reacting PBS with human serum was used as a negative control, and a ratio of the measured value of the negative control and a measured value in each reaction liquid was calculated by the following formula.

$$( Ratio with negative control ) = ( Measured value in each sample ) \div ( Measured value of negative control )$$

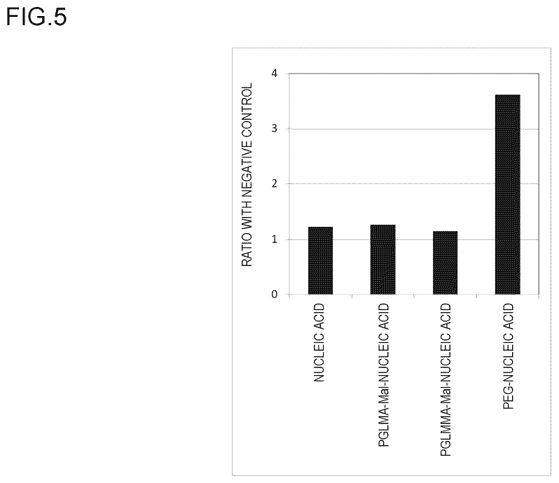

As a result, as shown in FIG. 5, in Conjugates 5 and 6, there was no large difference from the measured value of the oligonucleic acid as a comparative target, but it was found that in PEG-nucleic acid, stronger complement activation occurred than in the oligonucleic acid.

### Example 3: Preparation of Conjugate with Antibody

After 0.4 g of ammonium bicarbonate was dissolved in 80 mL of ultrapure water, formic acid was appropriately added until the pH reached 7.1, and then the solution was adjusted to 100 mL with ultrapure water, thereby preparing Conjugate buffer C. After 10 mg of an IgG antibody (normal human-derived, FUJIFILM Wako Pure Chemical Corporation) was dissolved in 1 mL of Conjugate buffer C, TCEP (tris(2-carboxyethyl) phosphine) hydrochloride was added so as to have a final concentration of 5 mM, and the mixture was reacted at room temperature for 30 minutes to prepare a reduced antibody having a thiol group. To this reduced antibody, Polymer 1 prepared in Production Example 1 was added in an amount of 5 molar equivalents of the antibody (Polymer 1:antibody=1:3.33 (mass ratio)), and the mixture was incubated at 25° C. for 1 hour to obtain a reaction product. After incubation, the reaction product was concentrated using an ultrafiltration unit (Amicon Ultra 50K device, Merck Corporation), then diluted with PBS, and concentrated again with the ultrafiltration unit. This operation was repeated three times, and unreacted Polymer 1 was removed to obtain Conjugate 7 (PGLMA-Mal-IgG) of the polymer and the reduced antibody. Also for Polymer 3 prepared in Production Example 3, Conjugate 8 (PGLMMA-Mal-IgG) with an IgG antibody was obtained by the same method as described above.

The outlines of Conjugates 1 to 8 produced in Examples 1 to 3 described above are listed in Table 1 below. “-Mal-” in the right column of Examples 1, 2, and 3 represents a bond including the structure of General Formula (1) described above, and “—Suc-” of Example 1 represents a bond including the structure of General Formula (3) described above.

TABLE 1

| | | Outline of configuration |
|---|---|---|
| | Conjugate number | of conjugate |
| Example 1 | Conjugate 1 | PGLMA-Mal-BSA |
| | Conjugate 2 | PGLMMA-Suc-BSA |
| | Conjugate 3 | PGLMMA-Mal-BSA |
| | Conjugate 4 | PGLMA-Suc-BSA |
| Example 2 | Conjugate 5 | PGLMA-Mal-nucleic acid |
| | Conjugate 6 | PGLMMA-Mal-nucleic acid |
| Example 3 | Conjugate 7 | PGLMA-Mal-IgG |
| | Conjugate 8 | PGLMMA-Mal-lgG |

### [Cytotoxicity Test of Terminal Functional Group-Introduced Compound (Polymer) and Conjugate Using Cultured Cells]

L929 cells (DS Pharma Biomedical Co., Ltd.), which are mouse-derived fibroblasts, were cultured in DMEM medium (Nacalai Tesque) added with fetal bovine serum (FBS) (DS Pharma Biomedical Co., Ltd.) at a final concentration of 10 w/v %. The cells were seeded on a 100 mm cell culture dish (BD Falcon) so as to have a size of 5.0×10³ cells/cm², and cultured under the conditions of 37° C. and 5% CO₂. The L929 cells cultured in the 100 mm cell culture dish to a 70% confluent state were treated with 0.25 w/v % trypsin/50 mM EDTA solution, and the above-mentioned serum-added DMEM medium was added to stop the trypsin reaction, thereby obtaining an L929 cell suspension. The number of cells in the L929 cell suspension was measured using a 0.4 w/v % trypan blue solution (FUJIFILM Wako Pure Chemical Corporation). The cell suspension was seeded on a 96-well plate (Thermo Fisher Science) so that the number of cells per well was 2.5×10³ cells, and cultured for 24 hours under the conditions of 37° C. and 5% CO₂. After 24 hours, 50 ML of the medium was removed from each well, 50 μL of a polymer solution in which the terminal functional group-introduced compounds (polymers) prepared in Production Examples 1 and 2 or the conjugates (PGLMA-Mal-BSA, PGLMA-Suc-BSA, PGLMA-Mal-nucleic acid, and PGLMA-Mal-IgG) prepared in Examples 1 to 3 were dissolved in PBS to 2 w/v % was added to each well, and the mixture was incubated for 24 hours under the conditions of 37° C. and 5% CO₂. After incubation, 51 μL of Cell Proliferation Kit II (XTT) (Merck Corporation) reagent was added to each well, and the cells were incubated for 3 hours under the conditions of 37° C. and 5% CO₂. Thereafter, the absorbance was measured with a plate reader SH-9000 (CORONA ELECTRIC Co., Ltd.). The measurement protocol conformed to the manual attached to the kit. The survival rate of L929 cells was calculated from the following formula based on the measured values of the wells tested by adding PBS instead of the polymer solution and the measured values of the wells to which each sample was added.

$$( Survival rate ) [ \% ] = ( Measured value of well with each sample added ) \div ( Measured value of well with PBS added ) \times 100$$

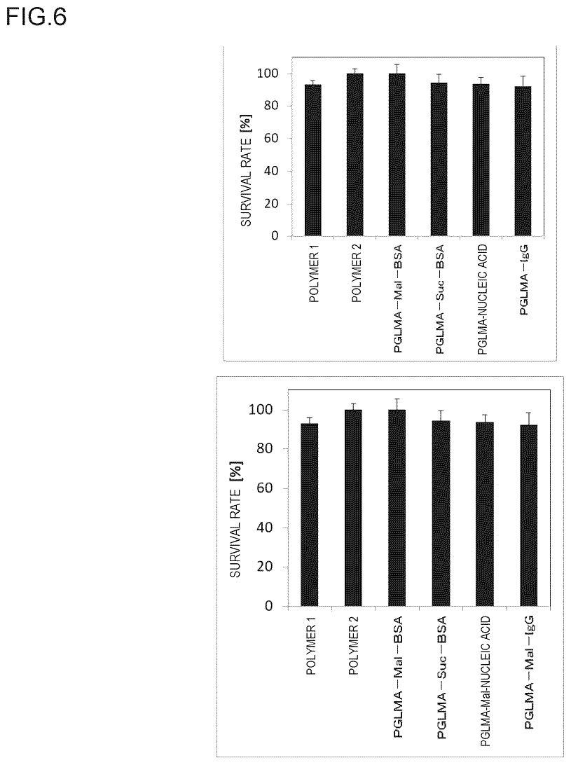

As a result, as shown in FIG. 6, none of the polymers and the conjugate solutions gave a significant difference in the survival rate of L929 cells, and no significant cytotoxicity was observed.

The present application is based on Japanese Patent Application No. 2021-202991 filed on Dec. 15, 2021, the disclosure content of which is incorporated herein by reference in its entirety.

1. A conjugate compound of a polymer (A) having a constitutional unit derived from a monomer (a) having two or more hydroxyl groups and having 2 to 10 carbon atoms constituting a side chain among carbon atoms of the constitutional unit, and a component (B) containing at least one selected from the group consisting of an amino acid, a polypeptide, a protein, a nucleoside, a nucleotide, and a nucleic acid.

2. The conjugate compound according to claim 1, wherein a content of the constitutional unit derived from the monomer (a) per 100 parts by mass of the polymer (A) is 5 parts by mass or more.

3. The conjugate compound according to claim 1, wherein the polymer (A) and the component (B) have at least one selected from the group consisting of an azide group, a thiol group, an amino group, an alkynyl group, a maleimide group, a succinimide group, a leaving group, and a disulfide group.

4. The conjugate compound according to claim 1, comprising at least one of bonds represented by General Formulas (1) to (4): 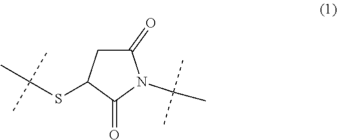 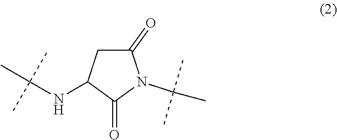 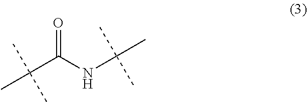 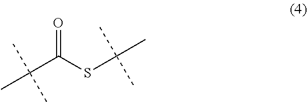
wherein dashed lines each indicate the attachment to the polymer (A) and the component (B).

5. The conjugate compound according to claim 1, wherein the constitutional unit derived from the monomer (a) includes a constitutional unit represented by General Formula (5): 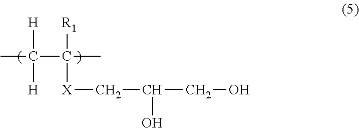
wherein R₁ represents a hydrogen atom or a methyl group, and X represents —C(═O)—O—, —C(═O)—NH—, —O—, —CH₂O—, or —CH₂CH₂O—.

6. The conjugate compound according to claim 1, wherein a number average molecular weight (Mn) of the polymer (A) is 1000 to 50000.

7. The conjugate compound according to claim 1, wherein a polydispersity Mw/Mn of the polymer (A) is 1.00 or more and 2.00 or less.

8. The conjugate compound according to claim 1, wherein a content mass ratio, the polymer (A)/the component (B), of the polymer (A) and the component (B) is 1/999 to 499/1.

9. A composition comprising the conjugate compound according to claim 1.

10. A pharmaceutical additive comprising the conjugate compound according to claim 1.

11. A method for producing a conjugate compound, comprising a step of reacting a polymer (A) having a constitutional unit derived from a monomer (a) having two or more hydroxyl groups and having 2 to 10 carbon atoms constituting a side chain among carbon atoms of the constitutional unit with a component (B) containing at least one selected from the group consisting of an amino acid, a polypeptide, a protein, a nucleoside, a nucleotide, and a nucleic acid.

12. The method for producing a conjugate compound according to claim 11, wherein the polymer (A) has a maleimide structure or a succinimide structure at a terminal, the component (B) has at least one selected from the group consisting of an azide group, a thiol group, an amino group, an alkynyl group, a maleimide group, a succinimide group, and a leaving group, and the maleimide structure or the succinimide structure is reacted with the at least one selected from the group consisting of an azide group, a thiol group, an amino group, an alkynyl group, a maleimide group, a succinimide group, and a leaving group.


SEQUENCE LISTING (1 sequence)

SEQ ID NO: 1 - DNA, 14 bp, synthetic construct
  source 1..14: mol_type = other DNA
tagcaccatggttt